(19) United States
(12) Patent Application Publication
(10) Pub. No.: US 2024/0307282 A1
(43) Pub. Date: Sep. 19, 2024

(54) THICKENING AGENT FOR PERSONAL CARE CLEANSING COMPOSITIONS

(71) Applicant: BASF SE, Ludwigshafen (DE)

(72) Inventors: Ben Chuan Zhu, Shanghai (CN); Fang Qin Lin, Shanghai (CN); Ting Ting Hu, Shanghai (CN); Ke Wang, Shanghai (CN); Ye Fan Wu, Shanghai (CN); Sen Yang, Shanghai (CN); Wei Song Qu, Shanghai (CN); Yi Zhou Zheng, Shanghai (CN)

(21) Appl. No.: 18/575,898

(22) PCT Filed: Jul. 5, 2022
(86) PCT No.: PCT/CN2022/103926

§ 371 Date: Jan. 2, 2024

(30) Foreign Application Priority Data
Jul. 6, 2021 (WO) PCT/CN2021/104780

(51) Int. Cl. A61K 8/42; A61K 8/44; A61Q 19/10

(52) U.S. Cl. CPC A61K 8/42; A61K 8/442; A61Q 19/10; A61K 2800/436; A61K 2800/48

(57) ABSTRACT

It relates to a thickening agent comprising an amidoamine of formula (I)and a N-acylamino acid salt of formula (II), which are as defined in the description. It also relates to sulfate-free personal care cleansing compositions containing the thickening agent.

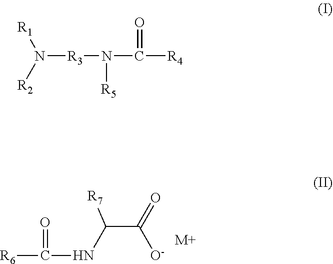

### FIELD OF THE INVENTION

The present invention relates to a thickening agent for sulfate-free personal care cleansing compositions and the sulfate-free personal care cleansing compositions containing the same.

### BACKGROUND

Personal care cleansing products, for example for face, body and hair, typically have aqueous formulations in form of shampoo, lotion, gel, liquid soap and wet wipes. Those formulations generally comprise thickening agents to provide sufficient consistency for applying on skin or hair. Various thickening agents were known useful for personal care cleansing products, for example inorganic salts or oxides such as sodium chloride, thickening or stabilizing polymers such as acrylic acid polymers and copolymers, naturally-derived gums, cellulose derivatives, cellulose-based materials, glycol esters or glycol ethers, alkanolamides such as fatty acid monoethanol amines, and alkyl glucosides.

Sulfate-based surfactants such as sodium lauryl sulfate and sodium lauryl ether sulfate are widely used in personal care cleansing products for their effectiveness in cleansing, foaming and stability. However, personal care compositions including sulfate-based surfactants present significant drawbacks. As a matter of fact, sulfate surfactants such as sodium laureth sulfate (SLS) are known to be liable to give rise to tolerance problems, especially on skin and eyes. Another drawback of sulfate surfactants is their tendency to strip oils, fats or proteins naturally contained on the surface of skin, scalp and hair. In the long term the repeated use of personal care compositions including sulfate-based surfactants may therefore cause irritation to the skin or scalp and/or give damage on hair fibers.

Recently, using non-sulfate surfactants becomes a trend in the field of personal care cleansing products. Such formulations have some drawbacks for example, poor foaming and poor clarity. Additionally, those formulations are difficult to thicken since non-sulfate surfactants such as N-acylglutamate generally contain a large hydrophilic head group. Many approaches have been proposed for personal care cleansing products to overcome the drawbacks accompanying the sulfate-free surfactants.

For example, WO2015089259A1 describes a sulfate-free personal care cleansing composition, which comprises a) water; b) up to about 10 wt %, based on the total weight of the personal care cleansing composition, of a surfactant selected from the group consisting of an anionic surfactant, an amphoteric surfactant, a nonionic/anionic surfactant mixture, and combinations thereof; c) a rheology modifying polymer; d) a cationic-substituted guar; and e) a copolymer of acrylamidopropyltrimonium chloride and acrylamide. As described in Examples of the patent application, the rheology modifying polymer based on cellulose and the combination of the cationic-substituted guar and the copolymer of acrylamidopropyltrimonium chloride and acrylamide are essential to provide desired viscosity of the formulation.

Thickening of the formulations containing non-sulfate surfactants remains a challenge. There is a need of novel and effective thickening agent for sulfate-free personal care cleansing formulations.

### SUMMARY OF THE INVENTION

It is an object of the present invention to provide a thickening agent for sulfate-free personal care cleansing products. Another object of the present invention is to provide sulfate-free personal care cleansing compositions which have desirable consistency.

It has been found that the objects of the present invention can be achieved by a combination of an amidoamine and a salt of N-acylamino acid, particularly a complex thereof.

Accordingly, in one aspect, the present invention relates to a thickening agent, which comprises
an amidoamine of formula (I)

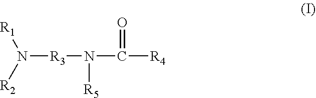

wherein
   R₁ and R₂, independently from each other, are H, or a straight-chain or branched C₁-C₆-alkyl,
   R₃ is a straight-chain or branched C₁-C₆-alkylene, and
   R₄ is a straight-chain or branched C₁₀-C₃₀-hydrocarbyl, and
   R₅ is H, or a straight-chain or branched C₁-C₆-alkyl, and
a N-acylamino acid salt of formula (II)

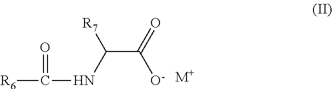

wherein
R₆ is a straight-chain or branched C₅-C₂₀-hydrocarbyl,
R₇ is a group which has a structure corresponding to a group bound to an a-carbon of an a-amino acid and containing no carboxyl substituent or protonable nitrogen-containing substituent, and
M⁺ is selected from alkali metal cation, quaternary ammonium cation and quaternary phosphonium cation.

In another aspect, the present invention relates to a personal care cleansing composition, which comprises
(A) at least one surfactant selected from the group consisting of non-sulfate anionic surfactant, amphoteric surfactant, nonionic surfactant, and any combinations thereof,
(B) a thickening agent as described herein, and
(C) water.

In a further aspect, the present invention relates to use of a combination of the amidoamine of formula (I) and the N-acylamino acid salt of formula (II), particularly a complex thereof, as a thickening agent in a sulfate-free aqueous cleansing composition.

### DETAILED DESCRIPTION OF THE INVENTION

The present invention now will be described in detail hereinafter. It is to be understood that the present invention may be embodied in many different ways and shall not be construed as limited to the embodiments set forth herein. Unless mentioned otherwise, all technical and scientific terms used herein have the same meaning as commonly understood by one of ordinary skill in the art to which the present invention belongs.

As used herein, the singular forms “a”, “an” and “the” include plural referents unless the context clearly dictates otherwise.

As used herein, the terms “comprise”, “comprising”, etc. are used interchangeably with “contain”, “containing”, etc. and are to be interpreted in a non-limiting, open manner. That is, e.g., further components or elements may be present. The expressions “consists of” or “consists essentially of” or cognates may be embraced within “comprises” or cognates.

As used herein, the term “aqueous” within the context of a formulation or a composition means that the solvent of the formulation or composition consists essentially of water or a combination of water and a water-soluble organic solvent. For example, at least 50% of the solvent in the formulation or composition is water.

As used herein, the terms “sulfate-free” within the context of the composition or formulation according to the present invention is to be understood that the composition or formulation does not comprise a significant amount of any sulfated compounds exerting a surfactant effect. In particular, here below to be understood that the sulfated compounds exerting a surfactant effect will be present in an amount of less than 0.01% based on the total weight of the composition or formulation, more particularly, in a non-detectable amount. The term “non-sulfate” within the context of a surfactant is to be understood that the surfactant is not any sulfated compound.

### <Thickening Agent>

A combination of amidoamine and N-acylamino acid was known useful for thickening an oily base, for example from U.S. Pat. No. 8,187,579B2. However, use of the combination of amidoamine and N-acylamino acid salt in an aqueous formulation and the effectiveness thereof for thickening an aqueous formulation were not described in U.S. Pat. No. 8,187,579 B2.

It has been surprisingly found that the combination of amidoamine and N-acylamino acid salt are effective for thickening aqueous formulations containing non-sulfate surfactants.

The present invention provides a thickening agent, which comprises
an amidoamine of formula (I)

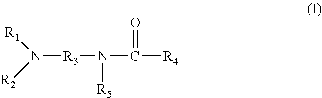

wherein
R₁ and R₂, independently from each other, are H, or a straight-chain or branched C₁-C₆-alkyl,
R₃ is a straight-chain or branched C₁-C₆-alkylene, and
R₄ is a straight-chain or branched C₁₀-C₃₀-hydrocarbyl, and
R₅ is H, or a straight-chain or branched C₁-C₆-alkyl, and
a N-acylamino acid salt of formula (II)

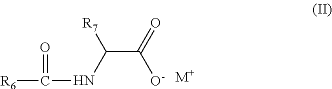

wherein
R₆ is a straight-chain or branched C₅-C₂₀-hydrocarbyl,
R₇ is a group which has a structure corresponding to a group bound to an a-carbon of an a-amino acid and containing no carboxyl substituent or protonable nitrogen-containing substituent, and
M⁺ is selected from alkali metal cation, quaternary ammonium cation and quaternary phosphonium cation.

The term “alkyl” as used herein refers to a saturated hydrocarbon radical, which may have for example 1 to ₆ (“C₁-C₆-alkyl”), 1 to 4 (“C₁-C₄-alkyl”) or ₁ to ₃ (“C₁-C₃-alkyl”) carbon atoms.

Examples of C₁-C₃-alkyl include methyl, ethyl, propyl and isopropyl. Examples of C₁-C₄-Alkyl additionally include n-butyl, 1-methylpropyl (sec-butyl), 2-methylpropyl (isobutyl) and 1,1-dimethylethyl (tert-butyl). Examples of C₁-C₆-alkyl further includes n-pentyl, 1-methylbutyl, 1-ethylpropyl, neo-pentyl, n-hexyl, 1-methylpentyl, 2-methylpentyl, 1-ethylbutyl, 2-ethylbutyl, etc.

The term “alkylene” as used herein refers to a divalent alkanediyl radical, which may have for example 1 to 6 carbon atoms (“C₁-C₆-alkylene”), 1 to 4 carbon atoms (“C₁-C₄-alkylene”) or 1 to 3 carbon atoms (“C₁-C₃-alkylene”). Examples of C₁-C₄-alkylene include, but are not limited to methylene, 1,2-ethylene, 1,3-propylene and 1,4-butylene. Examples of C₁-C₆-alkylene additionally include 1,5-pentylene, 1,6-hexylene, and isomers thereof.

The term “hydrocarbyl” as used herein refers to a radical of a saturated or unsaturated hydrocarbon, and may include, but are not limited to “alkyl” and “alkenyl”.

15 The term “acyl” as used herein refers to a straight-chain or branched hydrocarbyl that is bonded to the remainder of the compound directly via carbonyl, which may be represented by “hydrocarbyl-C(O)—”.

Amidoamines of formula (Ia) are particularly useful for the thickening agent according to the

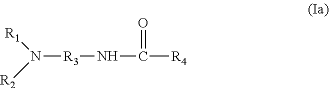

wherein
R₁ and R₂, independently from each other, are H, or a straight-chain or branched C₁-C₄-alkyl,
R₃ is a straight-chain C₁-C₄-alkylene, and
R₄ is a straight-chain or branched C₁₁-C₂₅-hydrocarbyl.

Preferably, the groups R₁ and R₂ in formula (Ia) are independently methyl or ethyl, preferably methyl.

Preferably, the group R₃ in formula (Ia) is a straight-chain C₁-C₃-alkylene, more preferably 1,3-propylene.

Preferably, the group R₄ in formula (la) is a straight-chain C₁₁-C₂₀-hydrocarbyl, preferably C15-C18-hydrocarbyl, more preferably C17-hydrocarbyl. Particularly, R₄ is a straight-chain C₁₁-C₂₀-alkyl, preferably C₁₅-C₁₈-alkyl, more preferably C₁₇-alkyl.

According to the present invention, the amidoamines of formula (Ia) wherein R₁ and R₂ are methyl, R₃ is 1,3-propylene and R₄ is a straight-chain C₁₅-C₁₈-alkyl is particularly preferred. More preferably, the amidoamine is stearamidopropyl dimethylamine.

The amidoamines may be prepared in accordance with known processes, for example by aminolysis of fatty acid alkylester with diamine. Commercially available amidoamines may also be used, for example those from BASF.

The N-acylamino acid salts useful for the thickening agent according to the present invention are particularly of formula (II) wherein R₆ is a straight-chain or branched C₇-C₁₇-hydrocarbyl, i.e., N—(C₈-C₁₈-acyl)amino acid salts, preferably of formula (II) wherein R₆ is a straight-chain or branched C₉-C₁₅-hydrocarbyl, i.e., N-(C₁₀-C₁₆-acyl)amino acid salts.

Examples of the acyl group in the N-acylamino acid salts, i.e., R₆—C(O)— in formula (II) may include, but are not limited to octanoyl, 2-ethylhexanoyl, nonanoyl, decanoyl, lauroyl, myristoyl, myristoleoyl, palmitoyl, palmitoleoyl, stearoyl, oleoyl, linoleoyl, cocoyl, and any combinations thereof. The acyl group is preferably octanoyl, 2-ethylhexanoyl, nonanoyl, decanoyl, lauroyl, myristoyl, myristoleoyl, palmitoyl or palmitoleoyl.

Suitable amino acids from which the N-acylamino acid salts are derived may include, but are not limited to glycine, alanine, valine, leucine, isoleucine, serine, threonine, cysteine, cystine, methionine, phenyl alanine and tyrosine. Preferably, the N-acylamino acid salts are selected from those derived from glycine, valine, cysteine and alanine, more preferably from glycine and alanine.

Preferably, the N-acylamino acid salts are alkali metal salts, for example lithium, sodium and potassium salts. In this case, M+in formula (II) is selected from Li⁺, Na⁺ and K⁺.

Alternatively, the N-acylamino acid salts may be quaternary ammonium and quaternary phosphonium salts. In this case, M⁺ in formula (II) may be cation of formula R′R″R″R″′N⁺ or R′R″R′'R″″P⁺ in which R′, R″, R′″ and R″″ are independently selected from H and optionally substituted C₁-C₄-alkyl. The C₁-C₄-alkyl may be substituted by for example OH or COOH.

The N-acylamino acid salts may be prepared in accordance with known processes, for example by Schotten-Bauman reaction of a fatty acid chloride with an amino acid under basic conditions. Commercially available acylamino acid salts may also be used, for example those from BASF.

According to the present invention, the amidoamine and the N-acylamino acid salt may be comprised in a molar ratio in the range of 0.7:1 to 1:0.2, preferably 1:1 to 1:0.2, more preferably in the range of 1:0.8 to 1:0.3, most preferably in the range of 1:0.7 to 1:0.4.

Particularly, the thickening agent comprises a complex of the amidoamine and the N-acylamino acid salt.

The thickening agent may be prepared simply by dissolving the amidoamine and the N-acylamino acid salt into water. The amidoamine and the N-acylamino acid salt may form a complex upon dissolution of both. The preparation may be carried out under stirring, and optionally heating, for example at a temperature in the range of 70 to 90° C., in order to facilitate the dissolution of the components and thus the formation of the complex.

The obtained mixture may be used as a thickening agent without any post-treatment. In this case, the thickening agent is in form of a composition comprising water, for example as a paste.

### <Personal Care Cleansing Composition>

In the second aspect, the present invention provides a personal care cleansing composition, which comprises
(A) at least one surfactant selected from the group consisting of non-sulfate anionic surfactant, amphoteric surfactant, nonionic surfactant and any combinations thereof,
(B) a thickening agent comprising
   an amidoamine of formula (I)

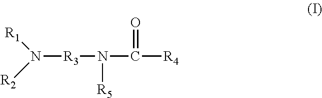

wherein
R₁ and R₂, independently from each other, are H, or a straight-chain or branched C₁-C₆-alkyl,
R₃ is a straight-chain or branched C₁-C₆-alkylene, and
R₄ is a straight-chain or branched C₁₀-C₃₀-hydrocarbyl, and
R₅ is H, or a straight-chain or branched C₁-C₆-alkyl,
a N-acylamino acid salt of formula (II)

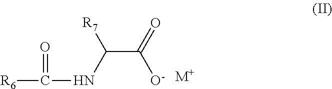

wherein
R₆ is a straight-chain or branched C₅-C₂₀-hydrocarbyl,
R₇ is a group which has a structure corresponding to a group bound to an a-carbon of an a-amino acid and containing no carboxyl substituent or protonable nitrogen-containing substituent, and
M⁺ is selected from alkali metal cation, quaternary ammonium cation and quaternary phosphonium cation,
and
(C) water.

Particularly, the personal care cleansing composition according to the present invention is sulfate-free. In other words, the surfactant in the personal care cleansing composition may consist of, or consist substantially of non-sulfate surfactants.

The sulfate-free personal care cleansing composition may comprise just one type of non-sulfate surfactant or a combination of different types of non-sulfate surfactant selected from nonionic surfactant, anionic surfactant, and amphoteric surfactant. For example, the sulfate-free personal care cleansing composition may comprise at least one anionic surfactant, or a combination of at least one nonionic surfactant and at least one anionic surfactant, or a combination of at least one nonionic surfactant and at least one amphoteric surfactant, or a combination of at least one anionic surfactant and at least one amphoteric surfactant.

In some embodiments, the personal care cleansing composition may comprise a combination of at least one nonionic surfactant, at least one anionic surfactant and at least one amphoteric surfactant.

There is no particular restriction to the non-sulfate surfactants for the purpose of the present invention. Any surfactants suitable for personal care cleansing compositions are applicable in the present invention.

Examples of nonionic surfactants useful for the personal care cleansing composition according to the present invention include, but are not limited to, polyethylene glycols, polyglycerol alkyl ethers, polyglycerol alkyl esters, ethoxylated derivatives of fatty alcohols, particularly C₈-C₁₆ fatty alcohols, ethoxylated derivatives of fatty acids, particularly C8-C16 fatty acids, ethoxylated derivatives of fatty acid esters, particularly C₈-C₁₆-fatty acid esters, ethoxylated derivatives of monoglycerides of C₈-C₁₆-fatty acids, ethoxylated derivates of sorbitan, ethoxylated derivates of mannitan, alkyl glycosides, alkyl polyglycosides, polyol glycosides, derivatives of ricin oil, polysorbates, fatty acid alkanolamide, N-alkylamines and amine oxides.

In some embodiments, the personal care cleansing composition according to the present invention comprises a nonionic surfactant selected from alkyl (poly)glycosides of formula (III),

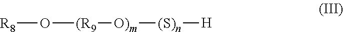

wherein
R₈ is a straight chain or branched C₆-C₂₄-hydrocarbyl,
R₉ is a straight-chain C₂-C₄-alkylene,
m is a number in the range of 0 to 10, preferably 0 to 4,
S is a remainder of a reducing sugar having 5 or 6 carbons, and
n is a number in the range of 1 to 5, preferably 1 or 2, and any combinations thereof.

In above formula (III), R₈ is preferably a straight chain or branched C₈-C₁₈-alkyl, for example n-octyl, n-nonyl, n-decyl, n-undecyl, n-dodecyl, n-tridecyl, n-tetradecyl, n-pentadecyl, n-hexadecyl, heptadecyl or octadecyl. Additionally, or alternatively, S is a remainder of a reducing sugar selected from glucose, fructose, galactose, xylose or arabinose, preferably glucose.

Preferably, in above formula (III), m is 0 and n is 1.

Examples of anionic surfactants useful for the personal care cleansing composition according to the present invention include, but are not limited to sulfonates such as alpha-olefin sulfonates and paraffin sulfonates, alkylphosphates, alkylether phosphates, alkylamido sulfonates, alkylaryl sulfonates, alkyl carboxylates, alkyl sulfosuccinates, alkylether sulfosuccinates, alkylamide sulfosuccinates, alkyl sulfoacetates, acyl isethionates, glycosyl carboxylates, alkyl polyglucoside derivatives such as citrates, tartrates, sulphosuccinates, carbonates and glycerol ethers obtained from alkyl (poly)glucosides, and N-acylamino acid salts. Those anionic surfactants may be ammonium salts, phosphonium salts or alkali metal salts.

In some embodiments, the personal care cleansing composition according to the present invention particularly comprises an alkyl (poly)glycoside derivative anionic surfactant. There may be mentioned in particular acetates, citrates, tartrates, sulphosuccinates, carbonates and glycerol ethers obtained from C₆-C₂₄-hydrocarbyl (poly)glucosides, especially salts of carboxylmethyl ether of C₆-C₂₄-hydrocarbyl (poly)glycosides.

Particularly, the personal care cleansing composition comprises an anionic surfactant selected from salts of carboxylmethyl ether of alkyl (poly)glycosides of (IV),

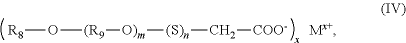

wherein
R₈ is a straight chain or branched C₆-C₂₄-hydrocarbyl,
R₉ is a straight-chain C₂-C₄-alkylene,
m is a number in the range of 0 to 10, preferably 0 to 4,
S is a remainder of a reducing sugar having 5 or 6 carbons,
n is a number in the range of 1 to 5, preferably 1 or 2,
M^{x+} is a quaternary ammonium cation, quaternary phosphonium cation, alkali metal cation or alkali earth metal cation, and
x is 1 or 2, equivalent to the positive charge of M,
and

any combinations thereof.

In above formula (IV), R₈ is preferably a straight chain or branched C₈-C₁₈ alkyl, preferably straight chain C₁₀-C₁₄ alkyl, for example n-octyl, n-nonyl, n-decyl, n-undecyl, n-dodecyl, n-tridecyl, n-tetradecyl, n-pentadecyl, n-hexadecyl, heptadecyl or octadecyl, more preferably n-dodecyl. Additionally or alternatively, S is a remainder of a reducing sugar selected from glucose, fructose, galactose, xylose or arabinose, preferably glucose.

Preferably, in above formula (IV), m is 0, n is 1 and x is 1.

In some other embodiments, the personal care cleansing composition comprises an anionic surfactant selected from N-acylamino acid based surfactants i.e., N-acylamino acid salts. Suitable N-acylamino acid salts may include, but are not limited to, N—(C₆-C₂₀-acyl)amino acid salts, N—(C₈-C₁₈-acyl)amino acid salts and N-(C10-C16-acyl)amino acid salts. Suitable amino acids from which the N-acylamino acid salts are derived may be selected from glycine, alanine, valine, leucine, isoleucine, methionine, proline, cysteine, phenyl alanine, tyrosine, tryptophan, arginine, lysine, histidine, aspartic acid, glutamic acid, serine, threonine and aspergine. Glutamic acid, aspartic acid, histidine and arginine may be particularly mentioned, since the corresponding N-acylamino acid salts are particularly difficult to thicken.

Particularly, the personal care cleansing composition comprises an anionic surfactant selected from N—(C₆-C₂₀-acyl)glutamates, preferably N-(C₈-C₁₈-acyl)glutamates, more preferably N—(C₁₀-C-₁₆-acyl)glutamates, among which N-cocoylglutamates may be particularly mentioned.

Examples of amphoteric surfactants useful for the personal care cleansing composition according to the present invention include, but are not limited to alkyl betaines such as C₈-C₂₀-alkylbetaines, alkylamido betaines such as (C₈-C₂₀-alkyl)amido(C₂-C₈-alkyl)betaines, sultaines, sulfobetaines alkylsulfobetaines, alkylamido alkylsulfo betaines such as (C₈-C₂₀-alkyl)amido(C₂-C₈-alkyl)sulfobetaines, derivatives of imidazolines, phosphobetaines, acyl taurate, amphoacetates and amphopropionates, preferably alkyl betaines, alkylamido betaines, sufobetaines, alkylsulfobetaines, alkylamido alkylsulfo betaines, amphoacetates, amphodiacetates, amphopropionates, amphodipropionates, and amphocarboxyglycinates.

In some embodiments, the personal care cleansing composition comprises an amphoteric surfactant selected from C₈-C₂₀-alkylbetaines and C₈-C₂₀-alkylamido-C₁-C₆-alkylbetaines, among which coco-betaine and cocamidopropylbetaine may be particularly mentioned.

In some other embodiments, the personal care cleansing composition comprises an amphoteric surfactant selected from amphoacetates and amphopropionates, particularlyy of formula (V)

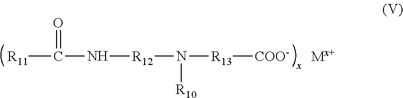

wherein
R₁₀ is a straight-chain or branched C₁-C₄-alkyl, optionally substituted with a hydroxyl group,
R₁₁ is a straight-chain or branched C₆-C₂₄-hydrocarbyl,
R₁₂ is a straight-chain C₂-C₆-alkylene,
R₁₃ is methylene or ethylene,
M^{x+} is a quaternary ammonium cation, quaternary phosphonium cation, alkali metal cation or alkali earth metal cation, and
x is 1 or 2, equivalent to the positive charge of M, and
any combinations thereof.

In above formula (V), R₁₁ is preferably a straight chain or branched C₈-C₁₈-alkyl, preferably straight chain C₁₀-C₁₄-alkyl, for example n-octyl, n-nonyl, n-decyl, n-undecyl, n-dodecyl, n-tridecyl, n-tetradecyl, n-pentadecyl, n-hexadecyl, heptadecyl or octadecyl. Additionally or alternatively, R₁₂ is preferably C₂-C₄-alkylene, more preferably ethylene. Additionally or alternatively, R₁₃ is preferably methylene. R10 is preferably a straight-chain C₁-C₄-alkyl substituted with a hydroxyl group, more preferably hydroxymethyl or hydroxyethyl.

Particularly, the personal care cleansing composition comprises an amphoteric surfactant selected from amphoacetates of formula (V) wherein R₁₁ is a straight chain C₁₀-C₁₄ alkyl, preferably n-undecyl, R₁₂ is C₂-C₄-alkylene, preferably ethylene, R₁₃ is methylene, R₁₀ is a straight-chain C₁-C₄-alkyl substituted with a hydroxyl group, preferably hydroxymethyl or hydroxyethyl, and M^{x+} is an alkali metal cation, among which sodium N-(2-hydroxyethyl)-N-(2-lauroylaminoethyl) glycinate (Sodium Lauroamphoacetate) and sodium N-(2-hydroxyethyl)-N-(2-cocoylaminoethyl) glycinate (Sodium Cocamphoacetate) may particularly be mentioned.

The personal care cleansing composition may comprise the nonionic surfactant, anionic surfactant, amphoteric surfactant or any combinations thereof in any conventional amounts, for example 5 to 50% by weight in total, preferably 5 to 30% by weight, more preferably 10 to 20% by weight, based on the total weight of the personal care cleansing composition.

The thickening agent useful for the personal care cleansing composition according to the present invention are as described hereinabove in the first aspect. Any general description of the thickening agent and preference thereof are incorporated here by reference.

The personal care cleansing composition may comprise the thickening agent in an amount of 1 to 8% by weight, preferably 1.5 to 5% by weight, more preferably 2 to 4% by weight, calculated as solid content, based on the total weight of the personal care cleansing composition.

Water is comprised in the personal care cleansing composition according to the present invention as a carrier. The amount of water may vary substantially, depending on the application form of the personal care cleansing composition. The personal care cleansing composition may further comprise an organic carrier in a minor amount, for example less than 50 wt %, particularly 20 wt % or less, or even 10 wt % or less, based on the total amount of carriers. Suitable organic carriers usefor the application to skin and/or hair may include C₁-C₃-alcohols, C₁-C₆-alkyl acetates and any combinations thereof. In some embodiments, at least one of acetone, hydrocarbons such as isobutane, hexane and decene, halogenated hydrocarbons and volatile silicones such as cyclomethicone may also comprised as a carrier.

The personal care cleansing composition may further comprise one or more adjuvant and/or additive typically used in personal care cleansing compositions. Examples of useful adjuvants and additives include, but are not limited to stabilizing agents, film-forming compounds, co-solvents, hydrotropic agents, pH adjusting agents, plasticisers, emulsifiers, opacifiers, pearling agents, superfatting agents, sequestering agents, chelating agents, antioxidants, perfumes, preservatives, mineral fillers or pigments, exfoliating particles and texture agents.

The personal care cleansing composition may optionally comprise one or more benefit agents that can provide a positive and/or beneficial effect to the substrate being cleaned, e.g. hair and skin. Examples of the benefit agents include, but are not limited to emollients, moisturizers, conditioners including skin conditioners or hair conditioners e.g. silicones such as volatile silicones, gums or oils, or non-amino silicones and mixtures thereof, mineral oils, esters (e.g. butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate and propylene glycol isostearate), animal fats including acetylated lanolin alcohols, lanolin, lard, mink oil and tallow, fatty acids and alcohols including behenic acid, palmitic acid, stearic acid, behenyl alcohol, cetyl alcohol, eicosanyl alcohol and isocetyl alcohol; vitamins and derivatives thereof such as vitamin B complex, including thiamine, nicotinic acid, biotin, pantothenic acid, choline, riboflavin, vitamin B6, vitamin B12, pyridoxine, inositol, carnitine, vitamins A,C,D,E,K, and derivatives thereof such as vitamin A palmitate, and pro-vitamins, e.g., panthenol (pro vitamin B5), panthenol triacetate and mixtures thereof; free-radical scavengers; natural or synthetic abrasives; dyes; hair coloring agents; bleaching agents including skin and hair bleaching agents; UV absorbers, such as benzophenone, bornelone, PABA (Para Amino Benzoic Acid), butyl PABA, cinnamidopropyl trimethyl ammonium chloride, disodium distyrylbiphenyl disulfonate, potassium methoxycinnamate; anti-UV agents, such as butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, octyl dimethyl PABA (padimate O), red petrolatum; antimicrobial agents; antibacterial agents, such as bacitracin, erythromycin, triclosan, neomycin, tetracycline, chlortetracycline, benzethonium chloride, phenol, parachlorometa xylenol (PCMX), triclocarban (TCC), chlorhexidine gluconate (CHG), zinc pyrithione, selenium sulfide; antifungal agents; melanin regulators; tanning accelerators; depigmenting agents, such as retinoids such as retinol, kojic acid and its derivatives such as, for example, kojic dipalmitate, hydroquinone and its derivatives such as arbutin, transexamic acid, vitamins such as niacin, vitamin C and its derivatives, azelaic acid, placertia, licorice, extracts such as chamomile and green tea, where retinol, kojic acid, and hydroquinone are preferred; skin lightening agents such as hydroquinone, catechol and its derivatives, ascorbic acid and its derivatives; skin coloring agents, such as dihydroxyacetone; liporegulators; weight-reduction agents; anti-acne agents; anti-seborrhoeic agents; anti-ageing agents; anti-wrinkle agents; keratolytic agents; anti-inflammatory agents; anti-acne agents, such as tretinoin, isotretinoin, motretinide, adapalene, tazarotene, azelaic acid, retinol, salicylic acid, benzoyl peroxide, resorcinol, antibiotics such as tetracycline and isomers thereof, erythromycin, anti-inflammatory agents such as ibuprofen, naproxen, hetprofen, botanical extracts such as alnus, arnica, artemisia capillaris, asiasarum root, calendula, chamomile, nidium, comfrey, fennel, galla rhois, hawthorn, houttuynia, hypericum, jujube, kiwi, licorice, magnolia, olive, peppermint, philodendron, salvia, sasa albomarginata, imidazoles such as ketoconazole and elubiol; refreshing agents; cicatrizing agents; vascular-protection agents; agents for the reduction of dandruff (anti-dandruff agent), seborrheic dermatitis, or psoriasis, such as pyrithione salts, being formed from heavy metals such as zinc, tin, cadmium, magnesium aluminum, sodium and zirconium, like zinc pyrithione, shale oil and derivatives thereof such as sulfonated shale oil, selenium sulfide, sulfur, salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, ciclopirox olamine, anti-psoriasis agents such as vitamin D analogs, e.g. calcipotriol, calcitriol, and tacaleitrol, vitamin A analogs such as esters of vitamin A, including vitamin A palmitate, retinoids, retinols, and retinoic acid, corticosteroids such as hydrocortisone, clobetasone, butyrate, clobetasol propionate; antiperspirants or deodorants, such as aluminum chlorohydrates, aluminum zirconium chlorohydrates; immunomodulators; nourishing agents; depilating agents, such as calcium thioglycolate, magnesium thioglycolate, potassium thioglycolate, strontium thioglycolate; agents for combating hair loss; reducing agents for permanent-waving; reflectants, such as mica, alumina, calcium silicate, glycol dioleate, glycol distearate, silica, sodium magnesium fluorosilicate; essential oils and fragrances, and optical brighteners.

It has been found that the personal care cleansing composition comprising the thickening agent as described herein exhibits a desirable consistency as characterized by viscosity, especially at a pH of 6 or less, preferably in the range of 4.5 to 6.0, more preferably in the range of 5.0 to 5.5.

Accordingly, in the third aspect, the present invention relates to use of the combination of
an amidoamine of formula (I)

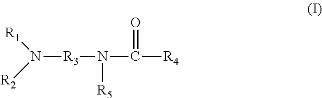

wherein
R₁ and R2, independently from each other, are H, or a straight-chain or branched C₁-C₆-alkyl,
R₃ is a straight-chain or branched C₁-C₆-alkylene, and
R₄ is a straight-chain or branched C₁₀-C₃₀-hydrocarbyl, and
R₅ is H, or a straight-chain or branched C₁-C₆-alkyl,
and
a N-acylamino acid salt of formula (II)

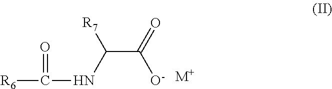

wherein
R₆ is a straight-chain or branched C₅-C₂₀-hydrocarbyl,
R₇ is a group which has a structure corresponding to a group bound to an a-carbon of a naturally occurring a-amino acid and containing no carboxyl substituent or protonable nitrogen-containing substituent, and
M+is selected from alkali metal cation, quaternary ammonium cation and quaternary
phosphonium cation, as a thickening agent in an aqueous sulfate-free cleansing composition.

The amidoamine of formula (I) and N-acylamino acid salt of formula (II) are as described hereinabove in the first aspect. Any general description and preference of the amidoamine of formula (I) and N-acylamino acid salt of formula (II) are incorporated here by reference.

The amidoamine of formula (I) and the N-acylamino acid salt of formula (II) is particularly useful for an aqueous sulfate-free cleansing composition having a pH in the range of 4.5 to 6.0, for example 5.0 to 5.5, especially for rinse-off personal care cleansing compositions, for example shampoo, lotion, gel or liquid soap, or in wet wipes.

The inventors of the present invention have surprisingly found that use of the combination of stearamidopropyl dimethylamine and potassium lauryl glycinate with at least one surfactant, can additionally achieve pearlescent effect, in particular under acidic pH condition. The combination of stearamidopropyl dimethylamine and potassium lauryl glycinate can be used in a personal care composition as pearlescent agent.

The invention will be further illustrated by following Examples, which set forth particularly advantageous embodiments. While the Examples are provided to illustrate the present invention, they are not intended to limit it.

### EXAMPLES

### Description of materials used in Examples:

Dehyquart® S18: stearamidopropyl dimethylamine (complete)

Plantapon® Amino KG-L: potassium cocoyl glycinate (30.0-35.0%) and water (65.0-70.0%)

Plantapon® LGC sorb: sodium lauryl glucose carboxylate (15-25%), lauryl glucoside (10-20%) and water (60-70%)

Plantapon® ACG 50: sodium cocoyl glutamate (40-50%) and water (43.5-45.5%)

Plantacare® 818 UP: coco-glucoside (50-60%) and water (40-50%)

Dehyton® PK45: cocamidopropyl betaine (44-46%) and water (54-56%) Dehyton® ML: sodium lauroamphoacetate (25-29%) and water (71-75%) Dehyton® DC-AS: Disodium Cocoamphodiacetate(46-51%) and water(49-54%)

Comperlan® 100C: cocamide MEA (100%)

Arlypon® F: laureth-2 (100%)

Lamesoft® PO65: coco-glucoside (20-40%), glyceryl oleate (20-40%) and water (32-35%)

Texapon® SFA: disodium 2-sulfolaruate (30-50%) and water (50-70%)

Plantapon® SF: sodium cocoamphoacetate (10-20%), glycerin (8-10%), lauryl glucoside (5-15%), sodium cocoyl glutamate (max. 5%), sodium lauryl glucose carboxylate (max. 5%) and water (40-60%)

Salcare® Super 7: aqueous solution of a diallyl dimethyl ammonium chloride and acrylamide copolymer

BioToLife: yeast ferment extract (30.0-50.0%) and water (50.0-70.0%).

The above materials are all commercially available from BASF.

Glydant Plus® Liquid, DMDM hydantoin and lodopropynyl Butylcarbamate (77%), commercially available from Lonza

Euxyl® PE 9010, preservative, commercially available from Schülke

### General process for preparing thickening agents

Dehyquart® S18 solid (INCI: stearamidopropyl dimethylamine) was added into Plantapon® Amino KG-L solution (INCI: potassium lauryl glycinate) in a certain amount of H₂O. The obtained mixture was heated to 80° C. under stirring until Dehyquart® S18 was fully dissolved. After cooling down to room temperature, a pearlizing paste of 30% solid content was obtained, which comprises a complex of stearamidopropyl dimethylamine and potassium lauryl glycinate.

### Measurement for thickening performance in aqueous surfactant bases

The thickening performance for each aqueous formulations as shown in Table 1 was characterized by viscosity at 23° C. as measured via Brookfield RVT viscometer with spindle 6 at 20 rpm. The test results are also summarized in Table 1.

TABLE 1

| Molar | | | | | | | | | |
|---|---|---|---|---|---|---|---|---|---|
| ratio | | | | | | | | | |
| Dehyquat ® | | Plantapon ® | Plantapon ® | | | | | | Viscosity |
| S18/ | Dehyquart ® | Amino | LGC | Plantapon ® | Plantacare ® | Dehyton ® | Dehyton ® | | at |
| Plantapon ® | S18 | KG | sorb | ACG 50 | 818 UP | PK45 | ML | Total | pH = 5 |
| Amino | (a.m. * | (a.m. * | (a.m. * | (a.m. * | (a.m. * | (a.m. * | (a.m. * | s.c. * | (mPa · |
| KG-L | wt %) | wt %) | wt %) | wt %) | wt %) | wt %) | wt %) | wt % | s) |
| 0:1 | 0 | 2.40 | 4.80 | | 3.20 | 1.60 | | 12.00 | 20 |
| 0.3:1 | 0.68 | 1.72 | 4.80 | | 3.20 | 1.60 | | 12.00 | 520 |
| 0.5:1 | 0.95 | 1.45 | 4.80 | | 3.20 | 1.60 | | 12.00 | 1860 |
| 0.7:1 | 1.15 | 1.25 | 4.80 | | 3.20 | 1.60 | | 12.00 | 3060 |
| 0.9:1 | 1.30 | 1.1 | 4.80 | | 3.20 | 1.60 | | 12.00 | 3960 |
| 1:0.5 | 1.74 | 0.66 | 4.80 | | 3.20 | 1.60 | | 12.00 | 4140 |
| 1:0 | 2.40 | 0.00 | 4.80 | | 3.20 | 1.60 | | 12.00 | 700 |
| 0:1 | 0 | 2.4 | | 3.20 | 3.20 | | 3.20 | 12.00 | 1040 |
| 0.3:1 | 0.68 | 1.72 | | 3.20 | 3.20 | | 3.20 | 12.00 | 1220 |
| 0.5:1 | 0.95 | 1.45 | | 3.20 | 3.20 | | 3.20 | 12.00 | 1820 |
| 0.7:1 | 1.15 | 1.25 | | 3.20 | 3.20 | | 3.20 | 12.00 | 2400 |
| 1:0.9 | 1.43 | 0.97 | | 3.20 | 3.20 | | 3.20 | 12.00 | 2420 |
| 1:0.7 | 1.57 | 0.83 | | 3.20 | 3.20 | | 3.20 | 12.00 | 3600 |
| 1:0.5 | 1.74 | 0.66 | | 3.20 | 3.20 | | 3.20 | 12.00 | 5880 |
| Comperlan ® | 2.4 | | | 3.20 | 3.20 | | 3.20 | 12.00 | 400 |
| 100C | | | | | | | | | |
| Arlypon ® | 2.4 | | | 3.20 | 3.20 | | 3.20 | 12.00 | 100 |
| F | | | | | | | | | |
| Lamesoft ® | 2.4 | | | 3.20 | 3.20 | | 3.20 | 12.00 | 140 |
| PO65 | | | | | | | | | |

* s.c.: solid content;
** a.m.: active matter.

It can be seen that the thickening agent comprising stearamidopropyl dimethylamine and potassium lauryl glycinate is able to thicken non-sulfate surfactants effectively, especially at a molar ratio of 0.7:1 or higher. The thicikening agent according to the present invention endows the aqueous surfactant bases with improved consistency compared with conventional thickening agents represented by Comperlan 100C, Arlypon F and Lamesoft PO65.

It has also been found that the thickening agent comprising the amidoamine of formula (I) and the N-acylamino acid salt of formula (II) in a ratio of 0.9 :1 or higher has a good compatibility with non-sulfate surfactants, as observed from an aqueous system comprising the surfactant Plantacare® 818 UP and the thickening agent comprising stearamidopropyl dimethylamine and potassium lauryl glycinate and.

### Application Examples

Various formulations useful for rinse-off personal care cleansing products were provided and evaluated for the appearance and viscosity, which comprise the thickening agents prepared with the above general process, designated as CatAnionic.

### 1. Rody Wash Formulation.

| | | % by | |
|---|---|---|---|
| Phase | Ingredient | weight | Function |
| A | Water | 69.58 | Carrier |
| | Edeta ® BD | 0.10 | Chelating agent |
| | CatAnionic (mol ratio: 1:0.7)* | 8.00 | Thickening agent |
| | Plantapon ® LGC sorb | 16.00 | Anionic surfactant |
| | Plantacare ® 818 UP | 5.82 | Non-ionic surfactant |
| | Dehyton ® PK 45 | 5.56 | Amphoteric surfactant |
| | Glydant Plus ® Liquid | 0.50 | Preservative |
| | 50 wt % citric acid | q.s. | pH Adjustment |

*ratio of Dehyquat ® S18/Plantapon ® Amino KG-L

Mix phase A and stir it well under 80° C.;
Cool down to 45° C., then add phase B, stir until uniform;
Adjust pH with citric acid;

Appearance: Viscous clear colourless liquid
pH value: 5.44 (5.0~5.5), 25° C.
Viscosity: 3200 mPa · s; Brookfield; RVT; spindle 6; 20 rpm; 23° C.

### 2. Shampoo Formulation:

| | | % by | |
|---|---|---|---|
| Phase | Ingredient | weight | Function |
| A | Water | 71.89 | Carrier |
| | Dehyquart ® Guar TC | 0.20 | Conditioning agent |
| B | Edeta ® BD | 0.10 | Chelating agent |
| | CatAnionic (mol ratio: 1:0.5)* | 8.00 | Thickening agent |
| | Plantapon ® ACG 50 | 6.40 | Anionic surfactant |
| | Plantacare ® 818 UP | 5.82 | Non-ionic surfactant |
| | Dehyton ® ML | 11.85 | Amphoteric surfactant |
| C | Sodium Benzoate | 0.40 | Preservative |
| | Euxyl ® PE 9010 | 0.40 | Preservative |
| | Lamesoft ® PO 65 | 0.50 | Humectant |
| D | 50 wt % citric acid | q.s. | pH Adjustment |

*ratio of Dehyquat ® S18/Plantapon ® Amino KG-L

Mix phase A and stir it well under 80° C.;
Add phase B, stir until uniform;
Cool down to 45°° C., then add phase C, stir until uniform;
Adjust pH with citric acid.

Appearance: Viscous translucent colorless liquid
pH value: 5.23 (5.0~5.5), 25° C.
Viscosity: 2600 mPa · s, Brookfield; RVT, spindle 6, 20 rpm, 23° C.

### 3. Shampoo Formulation

| | | % by | |
|---|---|---|---|
| Phase | Ingredient | weight | Function |
| A | Water | 73.45 | Carrier |
| | CatAnionic (mol ratio: 1:0.5)* | 8.00 | Thickening agent |
| | Dehyton ® PK 45 | 4 | Amphoteric surfactant |
| | Dehyton ® ML | 6.40 | Amphoteric surfactant |
| | Plantacare ® 818 UP | 5.82 | Non-ionic surfactant |
| | Plantapon ® ACG 50 | 6.4 | Anionic surfactant |
| B | BioToLife (Sophoropid) | 1 | Antidandruff agent |
| | Glydant Plus ® Liquid | 0.5 | Preservative |
| C | 50 wt % citric acid | q.s. | pH adjustment |

*ratio of Dehyquat ® S18/Plantapon ® Amino KG-L

Mix phase A and stir it well under 80° C.;
Cool down to 45°° C., then add phase B, stir until uniform;
Adjust pH with citric acid.

Appearance: Viscous clear yellowish liquid
pH value: 5.31 (5.0~5.5), 25° C.
Viscosity: 3200 mPa · s, Brookfield; RVT; spindle 6; 20 rpm; 23° C.

### 4. Soap Base Body Wash Formulation

| Phase | Ingredient | by weight % | Function |
|---|---|---|---|
| A | Water, demin. | 23.195 | Carrier |
| | Edeta ® BD | 0.1 | Complexing agent |
| | Glycerin | 5 | Humectant |
| | Potassium Hydroxide | 0.3 | Ph adjustment |
| B | Texapon ® SFA | 6 | Anioic surfactant |
| | Plantacare ® 818 UP | 18.5 | Nonionic surfactant |
| C | Plantapon ® SF | 38 | Hybrid surfactant |
| | Lamesoft ® PO 65 | 1.5 | Lipid layer enhancer |
| | Plantapon ® LC 7 | 2 | Conditioning agent |
| | Cutina ® AGS | 2 | Pearlizer |
| | Lauric Acid | 1.5 | Consistency agent |
| | Salcare Super 7 | 0.1 | Conditioning agent |
| | CatAnionic (mol ratio 1:0.7)* | 4.00 | Thickening agent |
| D | 50 wt % citric acid | 0.08 | pH adjustment |
| | Glydant Plus ® Liquid | 0.5 | Preservative |

*ratio of Dehyquat ® S18/Plantapon ® Amino KG-L

Mix phase B and stir it well under 70° C.,
Premix phase C under agitation, and add phase B into phase C,
Add phase A into the mixture of B+C, stir until uniform,
Under agitation, add phase D slowly until uniform below 45° C.

Appearance: Viscous yellowish liquid with pearl
pH value: 6.49 (6.0~6.5), 25° C.
Viscosity: 5050 mPa · s, Brookfield; RVT; spindle 6; 20 rpm; 23° C.

### Test of Pearlescent Effect

### Test group 1:

| Water | To 100% |
|---|---|
| Dehyquart S18 (a.m. * | 1.48% |
| wt %) | |
| Plantapon Amino KG-L | 0.92% |
| (a.m. * wt %) | |
| Surfactant ( a.m. * wt %) | 12% |

| | Surfactants | | | |
|---|---|---|---|---|
| | Plantapon ® | Dehyton ® | Dehyton ® | Dehyton ® |
| pH value | LGC Sorb ( | PK 45 | ML | DC-AS |
| 4.5 | Pearl effect | clear | Pearl effect | Pearl effect |
| 5.0 | clear | clear | clear | Pearl effect |
| 5.5 | clear | clear | clear | Pearl effect |
| 6.0 | clear | clear | clear | Pearl effect |
| 6.5 | clear | clear | clear | Slight pearl |
| | | | | effect |
| 7.0 | clear | clear | clear | Hazy |

a.m. * wt %: weight percentage of the active matter

Above result shows, when the combination of stearamidopropyl dimethylamine and potassium lauryl glycinate is used with various surfactants under acidic condition(pH<5), the pearl effect can be obviously observed at room temperature. For a mild surfactant, in particular, dehyton DC-AS (Disodium Cocoamphodiacetate), pearl effect will be observed in wider pH range which pH<7, pH<6 is more obvious.

Test group 2:

| Water | To 100% | | | | |
|---|---|---|---|---|---|
| Dehyquart S18 | 1.48% | | | | |
| Plantapon Amino KG-L | 0.92% | | | | |
| Plantapon ® LGC Sorb | 4% | | | | |
| (a.m. * wt %) | | | | | |
| Dehyton ® ML | 8% | | | | |
| (a.m. * wt %) | | | | | |
| pH value | 4.5 | 5.0 | 5.5 | 6.0 | 6.5 |
| Appearance | Pearl | clear | clear | clear | clear |
| | effect | | | | |
| Viscosity | 19470 | 31200 | 36200 | 32000 | 23730 |

| Water | To 100% | | | | |
|---|---|---|---|---|---|
| Dehyquart S18 | 1.48% | | | | |
| Plantapon Amino KG-L | 0.92% | | | | |
| Plantapon ® LGC Sorb | 4% | | | | |
| (a.m. * wt %) | | | | | |
| Dehyton ® DC-AS | 8% | | | | |
| (a.m. * wt %) | | | | | |
| pH value | 4.5 | 5.0 | 5.5 | 6.0 | 6.5 |
| Appearance | Pearl | Pearl | Slight | clear | clear |
| | effect | effect | Pearl | | |
| | | | effect | | |

| Water | To 100% | | | | |
|---|---|---|---|---|---|
| Dehyquart ® S18 | 1.48% | | | | |
| Plantapon ® Amino KG- | 0.92% | | | | |
| L | | | | | |
| Plantapon ® LGC Sorb | 8% | | | | |
| (a.m. * wt %) | | | | | |
| Dehyton ® PK 45 | 4% | | | | |
| (a.m. * wt %) | | | | | |
| pH value | 4.5 | 5.0 | 5.5 | 6.0 | 6.5 |
| Appearance | Pearl | clear | clear | clear | clear |
| | effect | | | | |
| Viscosity | 24000 | 33800 | 31470 | 20130 | 5900 |

a.m. * wt %: weight percentage of the active matter

Above formulation including combination of stearamidopropyl dimethylamine and potassium lauryl glycinate and one or more surfactants, provide pearl effect in particular under acidic condition (pH<5).

1. A thickening agent, which comprises
an amidoamine of formula (I) 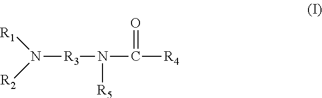
wherein
R₁ and R₂, independently from each other, are H, or a straight-chain or branched C₁-C₆-alkyl,
R₃ is a straight-chain or branched C₁-C₆-alkylene, and
R₄ is a straight-chain or branched C₁₀-C₃₀-hydrocarbyl, and
R₅ is H, or a straight-chain or branched C₁-C₆-alkyl,
and
a N-acylamino acid salt of formula (II) 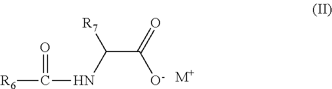
wherein
R₆ is a straight-chain or branched C₅-C₂₀-hydrocarbyl,
R₇ is a group which has a structure corresponding to a group bound to an α-carbon of an a-amino acid and containing no carboxyl substituent or protonable nitrogen-containing substituent, and
M⁺ is selected from the group consisting of alkali metal cation, quaternary ammonium cation and quaternary phosphonium cation.

2. The thickening agent according to claim 1, wherein the amidoamine is of formula (Ia), 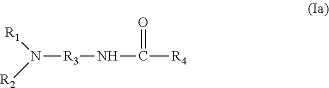
wherein
R₁ and R₂, independently from each other, are H, or a straight-chain or branched C₁-C₄-alkyl,
R₃ is a straight-chain C₁-C₄-alkylene, and
R₄ is a straight-chain or branched C₁₁-C₂₅-hydrocarbyl.

3. The thickening agent according to claim 1, wherein R₁ and R₂ are independently methyl or ethyl.

4. The thickening agent according to claim 1, wherein R₃ is a straight-chain C₁-C₃-alkylene.

5. The thickening agent according to any of preceding claim 1, wherein R₄ is a straight-chain C₁₁-C₂₀-hydrocarbyl.

6. The thickening agent according to claim 1, wherein in formula (II), R₆ is a straight-chain or branched C₇-C₁₇-hydrocarbyl.

7. The thickening agent according to claim 1, wherein the amino acid from which the N-acylamino acid salt is derived is selected from the group consisting of glycine, alanine, valine, leucine, isoleucine, serine, threonine, cysteine, cystine, methionine, phenyl alanine and tyrosine.

8. The thickening agent according to any of preceding claim 1, wherein the amidoamine and the N-acylamino acid salt are in a molar ratio in the range of 0.7: 1 to 1 : 0.2.

9. The thickening agent according to any of claim 1, wherein the amidoamine and the N-acylamino acid salt are present in form of a complex.

10. The thickening agent according to claim 1, wherein the thickening agent is in form of a composition comprising water.

11. A personal care cleansing composition, which comprises
(A) at least one surfactant selected from the group consisting of a non-sulfate anionic surfactant, amphoteric surfactant, nonionic surfactant, and any combination thereof,
(B) a thickening agent as defined in any of claims 1 to 10, and
(C) water.

12. The personal care cleansing composition according to claim 11, which is substantially free of a sulfate surfactant.

13. The personal care cleansing composition according to claim 11, which comprises the thickening agent in an amount of 1 to 8% by weight, calculated as solid content, based on the total weight of the personal care cleansing composition.

14. The personal care cleansing composition according to claim 11, wherein the nonionic surfactant comprises polyethylene glycols, polyglycerol alkyl ethers, polyglycerol alkyl esters, ethoxylated derivatives of fatty alcohols, particularly C₈-C₁₆ fatty alcohols, ethoxylated derivatives of fatty acids, ethoxylated derivatives of fatty acid esters, ethoxylated derivates of mannitan, alkyl glycosides, alkyl polyglycosides, polyol glycosides, derivatives of ricin oil, polysorbates, fatty acid alkanolamide, N-alkylamines, and amine oxides.

15. The personal care cleansing composition according to claim 11, which comprises a nonionic surfactant selected from alkyl (poly)glycosides of formula (III), 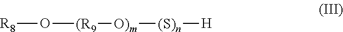
wherein
R₈ is a straight chain or branched C₆-C₂₄-hydrocarbyl,
R₉ is a straight-chain C₂-C₄-alkylene,
m is a number in the range of 0 to 10,
S is a remainder of a reducing sugar having 5 or 6 carbons, and
n is a number in the range of 1 to 5, and
any combination thereof.

16. The personal care cleansing composition according to claim 11, wherein the anionic surfactant comprises sulfonates, alkylphosphates, alkylether phosphates, alkylamido sulfonates, alkylaryl sulfonates, alkyl carboxylates, alkyl sulfosuccinates, alkylether sulfosuccinates, alkylamide sulfosuccinates, alkyl sulfoacetates, acyl isethionates, glycosyl carboxylates, alkyl (poly)glucoside derivatives. and N-acylamino acid salts.

17. The personal care cleansing composition according to claim 16, which comprises an alkyl (poly)glycoside derivative anionic surfactant, comprising acetates, citrates, tartrates, sulphosuccinates, carbonates and glycerol ethers obtained from C₆-C₂₄-hydrocarbyl (poly)glucosides.

18. The personal care cleansing composition according to claim 11, which comprises an N-acylamino acid salt anionic surfactant.

19. The personal care cleansing composition according to claim 11, wherein the amphoteric surfactant comprises alkyl betaines, alkylamido betaines, sultaines, sulfobetaines alkylsulfobetaines, alkylamido alkylsulfo betaines, derivatives of imidazolines, phosphobetaines, acyl taurate, amphoacetates and amphopropionates, preferably alkyl betaines, alkylamido betaines, sufobetaines, alkylsulfobetaines, alkylamido alkylsulfo betaines, amphoacetates, amphodiacetates, amphopropionates, amphodipropionates, and amphocarboxyglycinates.

20. The personal care cleansing composition according to claim 19, which comprises an amphoteric surfactant selected from C₈-C₂₀-alkylbetaines and C₈-C₂₀-alkylamido-C₁-C₆-alkylbetaines.

21. The personal care cleansing composition according to claim 19, which comprises an amphoteric surfactant selected from amphoacetates and amphopropionates.

22. (canceled)

23. A personal care cleansing composition comprising the amidoamine of formula (I) and the N-acylamino acid salt of formula (II) as defined in claim 1 wherein the composition is an aqueous sulfate-free cleansing composition.

24. The composition according to claim 23, wherein the aqueous cleansing composition has a pH in the range of 4.5 to 6.0.

25. A pearlescent personal care composition comprising the amidoamine of formula (I) and the N-acylamino acid salt of formula (II) as defined in claim 1.
